(19) United States
(12) Patent Application Publication
(10) Pub. No.: US 2013/0264238 A1
(43) Pub. Date: Oct. 10, 2013

(54) METHOD FOR PRODUCING A CONTAINER WELDED IN A PROTECTIVE COVER

(71) Applicant: Oliver Findl, Wien (AT)

(72) Inventor: Oliver Findl, Wien (AT)

(73) Assignee: CROMA-PHARMA GMBH, Leobendorf (AT)

(21) Appl. No.: 13/881,686

(22) PCT Filed: Aug. 12, 2010
(86) PCT No.: PCT/AT10/00296

§ 371 Date: May 14, 2013

(30) Foreign Application Priority Data
Sep. 3, 2009 (AT) A 1392/2009

(51) Int. Cl. B65B 55/14; A61F 9/00

(52) U.S. Cl. CPC B65B 55/14; A61F 9/0008
USPC 206/363; 53/425

(57) ABSTRACT

A method for producing a container welded in a protective wrapping, which contains a viscoelastic fluid for use as eye drops in the chirurgical treatment of eyes, wherein the container is configured such that the fluid may be removed from the container via a predetermined breaking point, characterized by the combination of the measures that

the viscoelastic fluid is filled into the container, which is thereupon closed,

the closed container is welded into a protective wrapping, whereupon

the welded container including the protective wrapping is subjected to thermal sterilization.

The invention relates to a method for producing a container welded in a protective wrapping, which container contains a fluid, which is used as a medicinal product in ophthalmic surgery. The invention further relates to the container welded in the protective wrapping, which is available via the method according to the invention.

In a surgical procedure the eye is usually wetted in regular intervals with a saline solution in order to prevent dehydration of the cornea. This process, however, is interrupting the work of the operating surgeon, is impairing the surgical progress and is destroying the homeostasis of the tear fluid. As a consequence, important components of the tear fluid such as, e.g., anti-inflammatory enzymes, lipids and mucopolysaccharides, are washed out.

It is further known to use viscoelastic fluids, so-called ophthalmic viscosurgical devices (OVD), filled into syringes also for continuous wetting and for epithelium protection of the cornea during certain ophthalmic surgery. The fluid is, e.g., a viscoelastic, physiologically acceptable formulation, which is used in order to protect the cornea against dehydration as well as against damage to the epithelium during eye surgeries, such as cataract surgeries, glaucoma surgeries, removal of foreign bodies or surgeries of the more posterior eye segment (posterior section surgeries such as e.g., vitrectomy, trabeculectomy). Thereby, the fluid is squashed out of a syringe and, if for optimal distribution necessary, is distributed onto the cornea using a spatula or a micro-sponge. The fluid has to be sterile.

Known fluids further contain preservatives, which, however, may be disadvantageous for the medical application. Also the external surface of the container, in which the fluid is contained, should further be sterile, thus facilitating handling by the surgeon.

According to U.S. Pat. No. 2,813,623 there are described elastic capsules containing the liquid drugs. The described packages are thereby sterilized by way of irradiation.

In EP 0 322 1345 welded small bottles having a liquid content, which may be used as eye drops, are described, wherein the small bottles have a screw-cap, through the opening of which the content may be extracted.

In U.S. Pat. No. 4,150,744 there are described containers for eye drops, which are contained in a shell, in which an oxygen-free atmosphere has been introduced. These containers have a dispenser, by means of which desired portions of the content of the container may be delivered, as well as a closing cap, by means of which the container may be re-closed upon discharging the desired drug portion.

In GB 1 275 903 there are disclosed packages, in which a drug is provided in a container having a removable (closing) cap.

Packaging and the production thereof according to prior art, however, do not always comply with the requirements posed to medicinal product packaging, which contain a viscoelastic fluid for use in the surgical treatment of eyes, and the production thereof.

Here, the present invention is positioned which aims at eliminating the above mentioned disadvantages or shortcomings, respectively.

That goal is accomplished by a method for producing a container welded in a protective wrapping, which contains a viscoelastic fluid for use in the surgical treatment of eyes, wherein the container is configured such that the fluid may be removed via a predetermined breaking point, which method is characterized by the combination of the measures that
the viscoelastic fluid is filled into the container, which is thereupon closed,
the closed container is welded into a protective wrapping, whereupon
the welded container including protective wrapping is subjected to thermal sterilization.

After the container has been welded into the protective wrapping, the internal as well as external sterility of the product is guaranteed via the terminal sterilization of the product. Another advantage of the method according to the invention is that no preservatives need be added to the viscoelastic fluid.

The container produced according to the invention guarantees better convenience for the surgeon when using it, as well as increased safety for the patient.

A preferred embodiment of the method according to the invention is, that the container is a single-dose container.

The container or single-dose container, respectively, is preferably made from polypropylene or mixtures of polyethylene or polypropylene with copolymers of ethylene and propylene, or from a laminate.

The protective wrapping consists preferably of a sterilizable medicinal paper and a composite film (e.g., Medipeel® Pouch by Sengewald) or Tyvek® material (manufacturer DuPont).

The thermal sterilization may be carried out at a temperature between 80 and 140° C.

The invention further relates to the container welded in a protective wrapping and producible according to the method according to the invention as such.

### DESCRIPTION OF THE FORMULATION

The formulation contains at least one viscosity increasing, physiologically acceptable polymer, which is known for wetting the eye surface, e.g., hydroxypropylmethyl cellulose, carboxymethyl cellulose, hydroxyethyl cellulose, hyaluronic acid, sodium alginate, hydroxylpropyl guar, polyvinylpyrrolidone (Povidone), polyvinyl alcohol, polymethacrylic acid (carbomer), polyethylene glycol.

The concentration preferably is 0.01-10%.

Furthermore, also combinations from two or several of these polymers be used.

As the fluid is introduced at the eye, the composition should have a pH value of 6 to 8.5, preferably 6.5 to 8, more preferably 6.8 to 7.6.

For the application of compositions at the eye, it is thus further advantageous if these have an osmolality comparable with that of the tear fluid. For this reason, the osmolality of the composition according to the invention is preferably 200 to 400 mosmol/l, even more preferably 250 to 330 mosmol/l.

The auxiliaries required thereby, such as, e.g., buffer salts, stabilizers, auxiliaries for adjusting the desired osmolality and auxiliaries for increasing acceptance are in each case dependent on the formulation and are well known to the skilled person.

### DESCRIPTION OF THE PACKAGING

The single-dose containers preferably consist of polypropylene (PP) having pharmaceutical quality. The polypropylene raw material used for the production of the single-dose containers has the following characteristics:

Melting point (determined according to ISO 3146): 100° C.-260° C.

Vicat softening temperature (10N, 50° C. per hour; determined according to ISO 306): 80° C.-240° C.;

Melt mass flow rate (230° C./2.16 kg; determined according to ISO 1133): 0.1 g/10 min-50 g/10 min;

Elongation (50 mm/min; determined according to ISO 527-2): 1%-30%;

Charpy notched impact strength (at 23° C.; determined according to ISO 179): 1 kj/m²-20 kj/m²

The protective wrapping for the single-dose container consists preferably of a sterilizable medicinal paper and a specialed composite film, wherein one side is transparent (e.g., Medipeel® Pouch by Sengewald) and provides for the external stability of the single-dose container.

### DESCRIPTION OF STERILIZATION

Due to the specialised packaging of the viscoelastic fluid in a single-dose container and a protective wrapping situated above, the internal and external sterility of the product is guaranteed by one single—terminal—sterilization step. The preferred type of sterilization is the physical sterilization by heat in a temperature range from 80° C.-140° C., e.g., by hot water sprinkling, saturated steam sterilization or sterilization by means of a steam-air mixture.

It has surprisingly turned out that the terminal sterilization by way of ionizing rays is not suitable in the present case, since this results in uncontrollable degradation of the viscosity increasing polymers in the fluid and the product following sterilization, hence, does no longer have the required viscoelastic characteristics. All other sterilization methods have the disadvantage that the sterilization cannot be carried out as terminal sterilization in the final container but rather two sterilization steps for internal and external sterility would be necessary.

### EXAMPLE OF A VISCOELASTIC FLUID

Water for injection purposes
Sodium hydroxide
Lactic acid
Sodium chloride
Potassium chloride
Calcium chloride×2H₂O
Hydroxypropylmethyl cellulose

Filling volume: 2 ml

pH: 6,8-7.6

Osmolality: 265-330 mosmol/kg

1. A method for producing a container welded in a protective wrapping, which contains a viscoelastic fluid for use as eye drops in the chirurgical treatment of eyes, wherein the container is configured such that the fluid may be removed from the container via a predetermined breaking point, characterized by the combination of the measures that
the viscoelastic fluid is filled into the container, which is thereupon closed,
the closed container is welded into a protective wrapping, whereupon
the welded container including the protective wrapping is subjected to thermal sterilization.

2. A method according to claim 1, wherein the container is a single-dose container suitable for the application of eye drops.

3. A method according to claim 1, wherein the container or single-dose container, respectively, is made from polypropylene or from mixtures of polyethylene or polypropylene with copolymers of ethylene and propylene or from a laminate.

4. A method according to claim 1, wherein the protective wrapping consists of a sterilizable medicinal paper and a composite film or Tyvek® material.

5. A method according to claim 1, wherein thermal sterilization is carried out at a temperature between 80 and 140° C.

6. A method according to claim 1, wherein the fluid is free from preservatives.

7. A container welded in a protective wrapping, producible by a method according to any of claims 1 to 6.
